(19) United States
(12) Patent Application Publication
(10) Pub. No.: US 2008/0034450 A1
(43) Pub. Date: Feb. 7, 2008

(54) Integration of commercial plant breeding and genomic technologies

(76) Inventor: Anna J. Frampton, Davis, CA (US)

Correspondence Address: BULLIVANT HOUSER BAILEY, 1415 L. STREET, SUITE 1000, SACRAMENTO, CA, 95814, US

(21) Appl. No.: 11/499,629

(22) Filed: Aug. 4, 2006

(51) Int. Cl. A01H 5/00

(52) U.S. Cl. 800/278; 800/279; 800/289

(57) ABSTRACT

The invention relates to a method for integration of commercial breeding of plants and genomic methodology comprising the steps of: a) plant population development by crossing a Parent 1 and a Parent 2 to generate a Population I; b) crossing Parent 1 with individuals from Population I to generated a Population II; c) crossing Parent 1 with individuals from population II to generate a Population III; d) randomly selecting at least one plant per each line in Population III and collecting genetic material from the random plant; e) self pollinating selected plants from population III to generate a Population IV; f) evaluating and selecting plants of Population IV; and g) using selecting progeny plants of Population IV in test crosses for evaluating the potential to develop new commercial cultivars; where the genetic material in step d) is used to develop marker profiles of each plant to map QTL and major gene loci as part of the evaluation of plants in step f).

### BACKGROUND OF THE INVENTION

1. Field of the Invention

The present invention relates to the field of plant breeding.

2. References
Abe H, Nakano M, Nakatsuka A, Nakayama M, Koshioka M, Yamagishi M. 2002. “Genetic analysis of floral anthocyanin pigmentation traits in Asiatic hybrid lily using molecular linkage maps.” Theor. Appl. Genet. 105:1175-118
Adam-Blondon A F, Roux C, Claux D, Butterlin G, Merdinoglu D, This P. 2004. “Mapping 245 SSR markers on the Vitis vinifera genome: a tool for grape genetics.” Theor. Appl. Genet. 109:1017-102
Alba R, Fei Z, Payton P, Liu Y, Moore S L, Debbie P, Cohn J, D'Ascenzo M, Gordon J S, Rose J K, Martin G, Tanksley S D, Bouzayen M, Jahn M M, Giovannoni J. 2004. “ESTs, cDNA microarrays, and gene expression profiling: tools for dissecting plant physiology and development.” Plant J. 39:697-71
Alm V, Fang C, Busso C S, Devos K M, Vollan K, Grieg Z, Rognli O A. 2003. “A linkage map of meadow fescue (Festuca pratensis Huds.) and comparative mapping of other Poaceae species.” Theor. Appl. Genet. 108:25-4
Asamizu E, Nakamura Y, Sato S, Tabata S. 2004. “Characteristics of the Lotus japonicus gene repertoire deduced from large-scale expressed sequence tag (EST) analysis.” Plant Mol. Biol. 54:405-41
Aubert G, Morin J, Jacquin F, Loridon K, Quillet M C, Petit A, Rameau C, Lejeune-Henaut I, Huguet T, Burstin J. 2006. “Functional mapping in pea, as an aid to the candidate gene selection and for investigating synteny with the model legume Medicago truncatula.” Theor. Appl. Genet. 112:1024-104
Babula D, Kaczmarek M, Barakat A, Delseny M, Quiros C F, Sadowski J. 2003. “Chromosomal mapping of Brassica oleracea based on ESTs from Arabidopsis thaliana: complexity of the comparative map.” Mol. Gen. Genomics 268:656-66
Bauer P, Thiel T, Klatte M, Bereczky Z, Brumbarova T, Hell R, Grosse I. 2004. “Analysis of sequence, map position, and gene expression reveals conserved essential genes for iron uptake in Arabidopsis and tomato.” Plant Physiol. 136:4169-418
Beedanagari S R, Dove S K, Wood B W, Conner P J. 2005. “A first linkage map of pecan cultivars based on RAPD and AFLP markers.” Theor. Appl. Genet. 110:1127-1137
Bednarek P T, Masojc P, Lewandowska R, Myskow B. 2003. “Saturating rye genetic map with amplified length polymorphism (AFLP) and random amplified polymorphic DNA (RAPD) markers.” J. App. Genet. 44:21-33
Bernacchi D, Tanksley S D. 1997. “An interspecific backcross of Lycopersicon esculentum x L. hirsutum: linkage analysis and a QTL study of sexual compatibility factors and floral traits.” Genetics 147:861-877
Blair M W, Pedraza F, Buendia H F, Gaitan-Solis E, Beebe S E, Gepts P, Thome J. 2003. “Development of genome-wide anchored microsatelite map for common bean (Phaseolus vulgaris L.)” Theor. Appl. Genet. 107:1362-1374
Bliss F A, Arulsekar S, Foolad M R, Becerra V, Gillen A M, Warburton M L, Dandekar A M, Kocsisne G M, Mydin K K. 2002. “An expanded genetic linkage map of Prunus based on an interspecific cross between almond and peach.” Genome 45:520-529
Bonnett D G, Rebetzke G J, Spielmeyer W. 2005. “Strategies for efficient implementation of molecular markers in wheat breeding”. Mol. Breeding 15:75-85
Bouchez A, Hospital F, Causse M, Gallais A, Charcosset A. 2002. “Marker-Assisted Introgression of Favorable Alleles at Quantitative Trait Loci Between Maize Elite Lines.” Genetics 162:1945-1959
Bowers J E, Abbey C, Anderson S, Chang C, Draye X, Hoppe A H, Jessup R, Lemke C, Lennington J, Li Z, Linn Y, liu S, Luo L, Marler B S, Ming R, Mitchel S E, Qiang D, Reischmann K, Schulze S R, Skinner D N, Wand Y, Kresovich S, Schertz K, Paterson H. 2003. “A High-Density Genetic Recombination Map of Sequence-Tagged Sites for Sorghum, as a Framework for Comparative Structural and Evolutionary Genomics of tropical Grains and Grasses. Genetics 165:367-386
Bradeen J M, Staub J E, Wye C, Antonise R, Peleman J. 2001. “Towards an expanded and integrated linkage map of cucumber (Cucumis sativus L).” Genome 44:111-119
Brugmans B, Hutten R G B, Rookmaker A N O, Visser R G F, van Eck H J. 2006. “Exploitation of a marker dense linkage map of potato for positional cloning of a wart disease resistance gene.” Theor. Appl. Genet. 112:269-277
Causse M, Saliba-Colombani V, Lesschaeve J, Buret M. 2001. “Genetic analysis of fruit quality in fresh market tomato. 2. Mapping QTLs for sensory attributes.” Theor. Appl. Genet. 102:273-283
Causse M, Saliba-Colombani V, Lecomte L, Duffe P, Rousselle P, Buret M. 2002. “QTL analysis of fruit quality in fresh market tomato: a few chromosome regions control the variation of sensory and instrumental traits.” J. Exp. Bot. 53:2089-2098
Cervera M T, Storme V, Ivens B, Gusmao J, Liu B H, Hostyn V, Van Slycken J, Van Montagu M, Boerjan W. 2001. “Dense Genetic Linkage Maps of Three Populus Species (Populus deltoides, P. nigra and P. trichocarpa) based on AFLP and Microsatellite Markers.” Genetics 158:787-809
Chaib J, Lecomte L, Buret M, Causse M. 2006. “Stability over genetic backgrounds, generations and years of quantitative trait locus (QTLs) for organoleptic quality in tomato.” Theor. Appl. Genet. 112:934-944
Charcosset A, Moreau L. 2004. “Use of molecular markers for the development of new cultivars and the evaluation of genetic diversity.” Euphytica 137:81-94
Chee P, Draye X, Jiang C-X, Decanini L, Delmonte T A, Bredhauer R, Smith C W, Paterson A H. 2005. “Molecular dissection of interspecific variation between Gossypium hirsutum and Gossypium barbadense (cotton) by a backcross-self approach: I. Fiber elongation.” Theor. Appl. Genet. 111:757-763
Chen M, Presting G, Barbazuk W B, Goicoechea J L, Blackmon B. Fang G, Kim H, Frisch D, Yu Y, Sun S, Higingbottom S, Phimphilai D, Thurmond S, Gaudette B, Li P, Liu J, Hatfield J, Main D, Farrar K, Henderson C, Barnett L, Costa R, Williams B, Wasler S, Atkins M, Hall C, Budiman M A, Tomkins J P, Luo M, Bancroft I, Salse J, Regard F, Mohapatra T, Singh N K, Tyagi A K, Soderlund C, Dean R A, Wing R A. 2002. “An Integrated Physical and Genetic Map of the Rice Genome.” The Plant Cell 14:537-545
Collard B C Y, Jahufer M Z Z, Brouwer J B, Pang E C K. 2005. “An introduction to markers, quantitative trait loci (QTL) mapping and marker-assisted selection for crop improvement: The basic concepts.” Euphytica 142:169-196
Coe E, Cone K, McMullen M, Chen S, Davis G, Gardiner J, Liscum E, Polacco M, Paterson A, Sanchez-Villeda H, Soderlund C, Wing R A. 2002. “Access to the Maize Genome: An Integrated Physical and Genetic Map.” Plant Physiol. 128:9-12
Cone C C, McMullen M D, Vroh Bi I, Davis G L, Yim Y-S, Gardiner J M, Polacco M L, Sanchez-Villeda H, Fang Z, Schroeder S G, Havermann S A, Bowers J E, Paterson A H, Soderlund C A, Engler F W, Wing R A, Coe Jr. E H. 2002. “Genetic, Physical, and Informatics Resources for Maize. On the Road to an Integrated map.” Plant Physiol. 130:1598-1605
Cregan P B, Jarvik T, Bush A L, Shoemaker R C, Lark K G, Kahler A L, Kaya N, VanToai T T, Lohnes D J, Chung J, Specht J E. 1999. “An integrated map of the soybean genome” Crop Sci. 39:1464-1490
Davis G L, McMullen M D, Baysdorfer C, Musket T, Grant D, Staebell L, Xu G, Polacco M, Koster L, Melia-Hancock S, Houchins K, Chao S, Coe Jr. E H. 1999. “A Maize Map Standard With Sequenced Core Markers, Grass Genome Reference Points and 932 Expressed Sequence Tagged sites (ESTs) in a 1736-Locus Map.” Genetics 152:1137-1172
De Koeyer D L, Tinker N A, Wight C P, Devl J, Burrows V D, O'Donoughue L S, Lybaert A, Molnar S J, Armtrong K C, Fedak G, Wesenberg D M, Rossnagel B G, McElroy A R. 2004. “A molecular linkage map with associated QTLs from a hulless x covered spring oat population.” Theor. Appl. Genet. 108:1285-1298
Dettori M T, Quarta R, Verde J. 2001. “A peach linkage map integrating RFLPs, SSRs, RAPDs, and morphological markers.” Genome 44:783-790
de Vicente M C, Tanksley S D. 1993. “QTL analysis of trangressive segregation in an interspecific tomato cross.” Genetics 134:585-596
Doganlar S, Frary A, Daunay M C, Lester R, Tanksley S D. 2002. “A comparative genetic linkage map of eggplant (Solanum melongena) and its implications for genome evolution in the Solanaceae.” Genetics 161:1697-1711
Doganlar S, Frary A, Daunay M C, Lester R, Tanksley S D. 2002. “Conservation of gene function in the solanaceae as revealed by comparative mapping of domestication traits in eggplant.” Genetics 161:1713-1726
Doganlar S, Frary A, Ku H-M, Tanksley S D. 2002. “Mapping quantitative trait loci in inbred backcross lines of Lycopersicon pimpinnellifolium (LA1589).” Genome 45:1189-1202
Doligez A, Bouquet A, Danglot Y, Lahogue F, Riaz S, Meredith P, Edwards J, This P. 2002. “Genetic mapping of grapevine (Vitis vinifera L.) applied to the detection of QTLs for seedlessness and berry weight.” Theor. Appl. Genet. 105:780-795
Drave X, Lin Y R, Qian X Y, Bowers J E, Burow G B, Morrell P L, Peterson D G, Presting G G, Ren S X, Wing R A, Paterson A H. 2001. “Toward integration of comparative genetic, physical, diversity, and cytomolecular maps for grasses and grains, using the sorghum genome as a foundation.” Plant Physiol. 125:1325-1341
Dreher K, Khairallah M, Ribaut J-M, Morris M. 2003. “Money matters (I): cost of field and laboratory procedures associated with conventional and marker-assisted maize breeding at CIMMYT.” Mol. Breeding 11:221-234
Dugo M L, Satovic Z, Millan T, Cubero J I, Rubiales D, Cabrera A, Torres A M. 2005. “Genetic mapping of QTLs controlling horticultural traits in diploid roses.” Theor. Appl. Genet. 111:511-520
Edwards M D, Stuber C W, Wendel J F. 1987. “Molecular-Marker-Facilitated Investigations of Quantitative-Trait Loci in Maize. I. Numbers, Genomic Distribution and Types of Gene Action.” Genetics 116:115-125
Eshed Y, Zamir D. 1995. “An introgression line population of Lycopersicon pennellii in the cultivated tomato enables the identification and fine mapping of yield associated QTL.” Genetics 141:1147-1162
Falque M, Decousset L, Dervins D, Jacob A M, Joets J, Martinant J P, Raffoux X, Ribiere N, Ridel C, Samson D, Charcosset A, Murigneux A. 2005. “Linkage Mapping of 1454 New Maize Candidate Loci.” Genetics 170:1957-1966
Fang Z, Polacco M, Chen S, Schroeder S, Hancock D, Sanchez H. Coe E. 2003(a). “cMap: the comparative genetic map viewer.” Bioinformatics 19:416-417
Fang Z, Cone K, Sanchez-Villeda H, Polacco M, McMullen M, Schroeder S, Gardiner J, Davis G, Havermann S, Yim Y, Vroh Bi I, Coe E. 2003(b). “iMap: a database-driven utility to integrate and access the genetic and physical maps of maize.” Bioinformatics 19:2105-2111
Faville M J, Vecchies A C, Schreiber M, Drayton M C, Hughes L J, Jones E S, Guthridge K M, Smith K F, Sawbridge T, Spanenberg G C, Bryan G T, Forster J W. 2004. “Functionally associated molecular genetic marker map construction in perennial ryegrass (Lolium perenne L.).” Theor. Appl. Genet. 110: 12-32
Feltus F A, Hart G E, Schertz K F, Casa A M, Kresovich S, Abraham S, Klein P E, Brown P J, Paterson A H. 2006. “Alignment of genetic maps and QTLs between inter and intra-specific sorghum populations.” Theor. Appl. Genet. 112:1295-1305
Fischer B M, Salakhutdinov I, Akkurt M, Eibach R, Edwards K J, Topfer R, Zyprian E M. 2004. “Quantitative trait locus analysis of fungal disease resistance factors on a molecular map of grapevine.” Theor. Appl. Genet. 108:501-515
Fourmann M, Barret P, Froger N, Baron C, Charlot F, Delourme R, Brunel D. 2002. “From Arabidopsis thaliana to Brassica napus: development of amplified consensus genetic markers (ACGM) for construction of a gene map.” Theor. Appl. Genet. 105:1196-1206
Francia E, Tacconi G, Crosatti C, Barabaschi D, Burgarelli D, Dall'Aglio E, Vale G. 2005. “Marker assisted selection in crop plants.” Plant Cell Tissue Organ Cult. 82:317-342
Frary A, Doganlar S, Daunay M C, Tanksley S D. 2003. “QTL analysis of morphological traits in eggplant and implications for conservation of gene function during evolution of solanaceous species.” Theor. Appl. Genet. 107:359-370
Frary A, Fulton T M, Zamir D, Tanksley S D. 2004. “Advanced backcross QTL analysis of a Lycopersicon esculentum x L. pennellii cross and identification of possible orthologs in the Solanaceae.” Theor. Appl. Genet. 108:485-496
Fridman E, Zamir D. 2003. “Functional divergence of a syntenic invertase gene family in tomato, potato, and Arabidopsis.” Plant Physiol. 131:603-609
Fulton T M, Van der Hoeven R, Eannetta N T, Tanksley S D. 2002. “Identification, analysis, and utilization of conserved ortholog set markers for comparative genomics in higher plants.” Plant Cell 14:1457-1467
Garcia A A, Kido E A, Meza A N, Souza H M, Pinto L R, Pastina M M, Leite C S, Silva J A, Ulian E C, Figueira A, Souza A P. 2006. “Development of an integrated genetic map of a sugarcane (Saccharum spp.) commercial cross, based on a maximum likelihood approach for estimation of linkage and linkage phases.” Theor. Appl. Genet. 112:298-314
Gedil M A, Wye C, Berry S, Segers B, Peleman J, Jones R, Leon A, Slabaugh M B, Knapp S J. 2001. “An integrated restriction fragment length polymorphism-amplified fragment length polymorphism linkage map of cultivated sunflower.” Genome 44:213-221
Gosselin I, Zhou Y, Bousquet J, Isabel N. 2002. “Megagametophyte-derived linkage maps of white spruce (Picea glauca) based on RAPD, SCAR and ESTP markers.” Theor. Appl. Genet. 104:987-997
Grant D, Cregan P, Shoemaker R C. 2000. “Genome organization in dicots: genome duplication in Arabidopsis and synteny between soybean and Arabidopsis.” Proc. Natl. Acad. Sci. USA 97:4168-4173
Hackett C A, Wachira F N, Paul S, Powell W, Waugh R. 2000. “Construction of a genetic linkage map for Camelia sinensis (tea).” Heredity 85:346-355
Hamwieh A, Udupa S M, Chuamane W, Sarker A, Drever F, Jung C, Baum M. 2005. “A genetic linkage map of Lens sp. based on microsatellite and AFLP markers and the localization of fusarium vascular wilt resistance.” Theor. Appl. Genet. 110:669-677
Hanley S, Barker A, Van Ooijen W, Aldam C, Harris L, Ahman I, Larsson S, Karp A. 2002. “A genetic linkage map of willow (Salix viminalis) based on AFLP and microsatellite markers.” Theor. Appl. Genet. 105:1087-1096
Hashizume T, Shimamoto I, Hirai M. 2003. “Construction of a linkage map and QTL analysis of horticultural traits for watermelon (Citrullus lanatus (THUNB.) MATSUM & NAKAI) usig RSAPD, RFLP and ISSR markers.” Theor. Appl. Genet. 106:779-785
Heckenberger M, Bohn M, Ziegle J S, Joe L K, Hauser J D, Hutton M, Melchinger A E. 2002. “Variation of DNA fingerprints among accessions within maize inbred lines and implications for identification of essentially derived varieties. I. Genetic and technical sources of variation in SSR data.” Mol. Breeding 10:181-191
Heckenberger M, van den Voort J R, Melchinger A E, Peleman J, Bohn M. 2003. “Variation of DNA fingerprints among accessions within maize inbred lines and implications for identification of essentially derived varieties. II. Genetic and technical sources of variation in AFLP data and comparison with SSR data.” Mol. Breeding 12:97-106
Heckenberger M, Muminovic J, van den Voort J R, Peleman J, Bohn M, Melchinger A E. 2006. “Identification of essentially derived varieties obtained from biparental crosses of homozygous lines. III. AFLP data from maize inbreds and comparison with SSR data.” Mol. Breeding 117:111-125
Hill W G. 1998. “Selection with recurrent Backcrossing to Develop Congenic Lines for Quantitative Trait Loci Analysis.” Genetics 148:1341-1352
Ho J C, McCouch S R, Smith M E. 2002. “Improvement of hybrid yield by advanced backcross QTL analysis in elite maize.” Theor. Appl. Genet. 105:440-448
Hori K, Kobayashi T, Shimizu A, Sato K, Takeda K, Kawasaki S. 2003. “Efficient construction of high-density map and its application to QTL analysis in barley.” Theor. Appl. Genet. 107:806-813
Hori K, Sato K, Nankaku N, Takeda K. 2005. “QTL analysis in recombinant chromosome substitution lines and doubled haploid lines derived from a cross between Hordeum vulgare ssp. vulgare and Hordeum vulgare ssp. spontaneum.” Mol. Breeding 16:295-311
Hospital F. 2005. “Selection in backcross programmes.” Phil. Trans R. Soc B 360:1503-1511
Huang X Q, Kempf H, Ganal M W, Roder M S. 2004. “Advanced backcross QTL analysis in progenies derived from a cross between a German elite winter wheat variety and a synthetic wheat (Triticum aestivum L.).” Theor. Appl. Genet. 109:933-943
Humphry E, Konduri V, Lambrides J, Magner T, McIntyre L, Aitken B, Liu J. 2002. “Development of a mungbean (Vigna radiata) RFLP linkage map and its comparison with lablab (Lablab purpureus) reveals a high level of colinearity between the two genomes.” Theor. Appl. Genet. 105:160-166
Ilic K, SanMiguel P J, Bennetzen J L. 2003. “A complex history of rearrangement in an orthologous region of the maize, sorghum, and rice genomes.” Proc. Natl. Acad. Sci USA 100:12265-12270
Isobe S, Klimenko I, Ivashuta S, Gau M, Kozlov N N. 2003. “First RFLP linkage map of red clover (Trifolium pretense L.) based on cDNA probes and its transferability to other red clover germplasm.” Theor. Appl. Genet. 108:105-112
Joobeur T, Periam N, de Vicente M C, King G J, Arus P. 2000. “Development of a second generation map for almond using RAPD and SSR markers.” Genome 43:649-655
Julier B, Flajoulot S, Barre P, Cardinet G, Santoni S, Huguet T, Huyghe C. 2003. “Construction of two genetic linkage maps in cultivated tetraploid alfalfs (Medicago sativa) using microsatelite and AFLP markers.” BMC Plant Biol 3:9 http://www.biomedcentral.com/147-2229/3/9
Keim P, Diers B W, olson T C, Shoemaker R C. 1990. “RFLP Mapping in Soybean: Association Between Marker Loci and Variation in Quantitative traits. Genetics 126:735-742
Kenis K, Keulemans J. 2005. “Genetic linkage maps of two apple cultivars (Malus x domestica Borkh.) based on AFLP and microsatellite markers.” Mol. Breeding 15:205-219
Kesseli R V, Paran I. Michelmore R W. 1994. “Analysis of a Detailed Genetic Linkage Map of Lactuca sativa (Lettuce) Constructed from RFLP and RAPD Markers.” Genetics 136:1435-1446
Korff von M, Wang H, Leon J, Pillen K. 2005. “AB-QTL analysis in spring barley. I. Detection of resistance genes against powdery mildew, leaf rust and scald introgressed from wild barley.” Theor. Appl. Genet. 111:583-590
Kremer C A, Lee M, Holland J B. 2001. “A restriction length fragment polymorphism based linkage map of a diploid Avena recombinant inbred line population.” Genome 44:192-204
Ku H M, Vision T, Liu J, Tanksley S D. 2000. “Comparing sequenced segments of the tomato and Arabidopsis genomes: large-scale duplication followed by selective gene loss creates a network of synteny.” Proc. Natl. Acad. Sci. USA 97:9121-9126
Ku H M, Liu J, Doganlar S, Tanksley S D. 2001. “Exploitation of Arabidopsis-tomato synteny to construct a high-resolution map of the ovate-containing region in tomato chromosome 2.” Genome 44:470-475
Kuchel H, Ye G, Fox R, Jefferies S. 2005. “Genetic and economic analysis of a targeted marker-assisted wheat breeding strategy.” Mol. Breeding 16:67-78
Lander E S, Botstein D. 1989. “Mapping Mendelian Factors Underlying Quantitative Traits Using RFLP Linkage Maps.” Genetics 121:185-199
la Rosa R, Angiolillo A, Guerrero C, Pellegrini M, Rallo L, Besnard G, Berville A, Martin A, Baldoni L. 2003. “A first linkage map of olive (Olea europaea L.) cultivars using RAPD, AFLP, RFLP and SSR markers.” Theor. Appl. Genet. 106:1273-1282
Lee M. 1998. “Genome projects and gene pools: New germplasm for plant breeding?” Proc. Natl. Acad. Sci USA 95:2001-2004
Levi A, Thomas E, Joobeur T, Zhang X, Davis A. 2002. “A genetic linkage map for watermelon derived from a testcross population: (Citrullus lanatus var. citroides x C. lanatus var. lanatus) x Citrullus colocynthis. Theor. Appl. Genet. 105:555-563
Liebhard R, Koller B, Gianfranceschi L, Gessler C. 2003. “Creating a saturated reference map for the apple (Malus x domestica Borkh.) genome.” Theor. Appl. Genet. 106:1479-1508
Lin C, Mueller L A, McCarthy J, Crouzillat D, Petiard V, Tanksley S D. 2005. “Coffee and tomato share common gene repertoires as revealed by deep sequencing of seed and cherry transcripts.” Theor. Appl. Genet. 112:114-130
Livingstone K D, Lackney V K, Blauth J R, van Wijk R, Kyle Jahn M. 1999. “Genome mapping in Capsicum and the evolution of genome structure in the Solanaceae.” Genetics 152:1183-1202
Liu Z H, Anderson J A, Hu J, Friesen T L, Rassmusen J B, Faris J D. 2005. “A wheat intervarietal genetic linkage map based on microsatelite and target region amplified polymorphism markers and its utility for detecting quantitative trait loci.” Theor. Appl. Genet. 111:782-794
Lodhi M A, Daly M J, Ye G N, Weeden N F, Reisch B I. 1995. “A molecular marker based linkage map of Vitis.” Genome 38:786-794
Manly K F, Cudmore R H, Meer J M. 2001. “Map Manager Q TX, cross-platform software for genetic mapping.” Mammalian Genome 12:930-932
Masi P, Spagnoletti Zeuli P L, Donini P. 2003. “Development and analysis of multiplex microsatellite marker sets in common bean (Phaseolus vulgaris L).” Mol. Breeding 11:303-313
Meaburn E, Butcher L M, Schalkwyk L C, Plomin R. 2006. “Genotyping pooled. DNA using 100K SNP microarrays: a step towards genomwide association scans.” Nuc. Acid Res. 34:e27
Mehlenbacher S A, Brown R N, Nouhra E R, Gokirmak T, Bassil N V, Kubisiak T L. 2006. “A genetic linkage map for hazelnut (Corylus avellana L.) based on RAPD and SSR markers.” Genome 49:122-133
Mei H W, Xu J L, Li Z K, Yu X Q, Guo L B, Wang Y P, Ying C S, Luo L J. 2006. “QTLs influencing panicle size detected in two reciprocal introgressive line (IL) populations in rice (Oryza sativa L.) Theor. Appl. Genet. 112:648-656
Menz M A, Klein R R, Mullet J E, Obert J A, Unruh N C, Klein P E. 2002. “A high-density genetic map of Sorghum bicolor (L) Moench based on 2926 AFLP, RFLP, and SSR markers.” Plant Mol. Biol. 48:483-499
Merdinoglu D, Butterlin G, Bevilacqua L, Chiquet V, Adam-Blondon A F, Decroocq S. “Development and characterization of a large set of microsatellite markers in grapevine (Vitis vinifera L.) suitable for multiplex PCR.” Mol. Breeding 15:349-366
Michelmore R W, Paran I, Kesseli R V. 1991. “Identification of markers linked to disease-resistance genes by bulkrd segregant analysis: A rapid method to detect markers in specific genomic regions by using segregating populations.” Proc. Natl. Acad. Sci. USA 88:9828-9832
Mohring S, Salamini S, Schneider K. 2004. “Multiplexed, linkage group-specific SNP marker sets for rapid genetic mapping and fingerprinting of sugar beet (Beta vulgaris L.).” Mol. Breeding 14:475-488
Moncada P, Martinez C P, Borrereo J, Chatel M, Gauch Jr. H, Guimaraes E, Thome J, McCouch S R. 2001. “Quantitative trait loci for yield and yield components in an Oryza sativa x Oryza rufipogon BC2F2 population evaluated in an upland environment.” Theor. Appl. Genet. 102:41-52
Monforte A J, Tanksley S D. 2000. “Development of a set of near isogenic and backcross recombinant inbred lines containing most of the Lycopersicon hirsutum genome in a L. esculentum background: a tool for gene mapping and gene discovery.” Genome 43:803-813
Monforte A J, Friedman E, Zamir D, Tanksley S D. 2001. “Comparison of a set of allelic QTL-NILs for chromosome 4 of tomato: Deductions about natural variation and implications for germplasm utilization.” Theor. Appl. Genet. 102:572-590
Moran J I, Bolton A D, Tran P V, Brown A, Dwyer N, Manning D K, Bjork B C, Li C, Montgomery K, Siepka S M, Hotz Vitaterna M, Takahashi J S, Wiltshire T, Kwiatkowski D J, Kucherlapati R, Beier D R. 2006. “Utilization of whole genome SNP panel for efficient genetic mapping in the mouse.” Genome Res. 16:436-440
Morris M, Dreher K, Ribaut J M, Khairallah M. 2003. “Money matters (II): costs of maize inbred line conversion schemes at CIMMYT using conventional and marker-assisted selection.” Mol. Breeding 11:235-247
Namkoong G, Lewontin R C, Yanchuk A D. 2004. “Plant genetic resource management: the next investments in quantitative and qualitative genetics.” Genetic Resources and Crop Evolution 51:853-862
Nelson J C. 1997. “QGENE: software for marker-based genomic analysis and breeding.” Mol. Breeding 3:239-245
Ogundiwin E A, Berke T F, Massoudi M, Black L L, Huestis G, Choi D, Lee S, Prince J P. 2005. “Construction of 2 intraspecific linkage maps and identification of resistance QTLs for Phytophthora capsici root-rot and foliar-blight diseases of pepper (Capsicum annuum L.).” Genome 48:698-711
Oliver M, Garcia-Mas J, Cardus M, Pueyo N, Lopez-Sese A L, Arroyo M, Gomez-Paniagua H, Arus P, de Vicente M C. 2001. “Construction of a reference linkage map for watermelon.” Genome 44:836-845
Ouedraogo J T, Gowda B S, Jean M, Close T J, Ehlers J D, Hall A E, Gillaspie A G, Roberts P A, Ismail A M, Bruening G, Gepts P, Timko M P, Belzile F J. 2002. “An improved genetic linkage map for cowpea (Vigna unguiculata L.) combining AFLP, RFLP, RAPD, biochemical markers, and biological resistance traits.” Genome 45:175-188
Paillard S, Schnurbusch T, Winzeler M, Messmer M, Sourdille P, Abderhalden O, Keller B, Schachermayr G. 2003. “An integrative genetic linkage map of winter wheat (Triticum aestivum L.).” Theor. Appl. Genet. 107:1235-1242
Pan Q, Lin Y S, Budai-Hadrian O, Sela M, Carmel-Goren L, Zamir D, Fluhr R. 2000. “Comparative genetics of nucleotide binding site—leucine rich repeat resistance gene homologues in the genomes of two dicotyledons: tomato and Arabidopsis.” Genetics 155:309-322
Paran I, Voort van den J R, Lefebvre V, Jahn M, Landry L, Schriek van M, Tanyolac B, Caranta C, Ben Chaim A, Livingstone K, Palloix A, Peleman J. 2004. “An integrated genetic linkage map of pepper (Capsicum spp.).” Mol. Breeding 13:251-261
Park Y H, Sensoy S, Wye C, Antonise R, Peleman J, Havey M J. 2000. “A genetic map of cucumber composed of RAPDs, RFLPs, AFLPs, and loci conditioning resistance to papaya ringspot and zucchini yellow mosaic viruses.” Genome 43:-01
Paterson A H, Lander E S, Hewitt J D, Peterson S, Lincoln S E, Tanksley S D. 1988. “Resolution of quantitative traits into Mendelian factors by using a complete linkage map of restriction fragment length polymorphisms.” Nature 335:721-726
Paterson A H, DeVema J W, Lanini B, Tanksley S D. 1990. “Fine Mapping of Quantitative Trait Loci Using Selected Overlapping Recombinant Chromosomes, in an Interpecific Cross of Tomato.” Genetics 124:735-742
Paterson A H, Damon S, Hewitt J D, Zamir D, Rabinowitch H D, Lincoln S E, Lander E S, Tanksley S D. 1991. “Mendelian Factors Underlying Quantitative Traits in Tomato: Comparison Across Species, Generations, and Environments.” Genetics 127:181-197
Pearl H M, Nagai C, Moore P H, Steiger D L, Osgood R V, Ming R. 2004. “Construction of a genetic map for Arabica coffee.” Theor. Appl. Genet. 108:829-835
Peleman J D, Wye C, Zethof J, Sorensen A P, Verbakel H, Oeveren van J, Gerats T, Voort van der J R. 2005. “Quantitative trait Locus (QTL) Isogenic Recombinant Analysis: A Method for High-Resolution Mapping of QTL Within a Single Population.” Genetics 171.1341-1352
Perin C, Hagen S, De Conto V, Katzir N, Danin-Poleg Y, portnoy V, dracco-Arnas S, Chadoeuf J, Dogimont C, Pitrat M. 2002. “A reference map of Cucumis melo based on two recombinant inbred line populations. Theor. Appl. Genet. 104:1017-1034.
Piquemal J, Cinquin E, Couton F, Rondeau C, Seignoret E, Doucet I, Perret D, Villeger M J, Vincourt P, Blanchard P. 2005. “Construction of an oilseed rape (Brassica napus L.) genetic map with SSR markers.” Theor. Appl. Genet. 111:1514
Pradhan A K, Gupta V, Mukhopadhyay A, Arumugam N, Sodhi Y S, Pental D. 2003. “A high-density linkage map in Brassica juncea (Indian mustard) using AFLP and RFLP markers.” Theor. Appl. Genet. 106: 607-614
Pugh T, Fouet O, Risterucci A M, Brottier P, Abouladze M, Deletrez C, Courtois B, Clement D, Larmande P, N'Goran J A, Lanaud C. 2004. “A new cacao linkage map based on codominant markers: development and integration of 201 new microsatellite markers.” Theor. Appl. Genet. 108:1151-1161
Quarrie S A, Steed A, Calestani C, Semikhodskii A, Lebreton C, Chinoy C, Steele N, Pljevljakusic D, Waterman D, Weyen J, Schondelmaier J, Habash D Z, Farmer P, Saker L, Clarkson D T, Abugalieva A, Yessimbekova M, Turuspekov Y, Abugalieva S, Tuberosa R, Sanguineti M C, Hollington P A, Aragues R, Royo A, Dodig D. 2005. “A high-density genetic map of hexaploid wheat (Triticum aestivum L.) from the cross Chinese Spring x SQ1 and its use to compare QTLs for grain yield across a range of environments.” Theor. Appl. Genet. 110:865-80
Ramsay L, Macaulay M, Ivanissevich S, MacLean K, Carlde L, Fuller J, Edwards K J, Tuvesson S, Morgante M, Massari A, Maestri E, Marmiroli N, Sjakste T, Ganal M, Powell W, Wough R. 2000. “A Simple Sequence Repeat-Based Linkage map of Barley.” Genetics 156:1997-2005
Rensink W A, Lee Y, Liu J, Iobst S, Ouyang S, Buell C R. 2005. “Comparative analyses of six solanaceous transcriptomes reveal a high degree of sequence conservation and species-specific transcripts.” BMC Genomics 6:124
Reyna N, Sneller C H. 2001. “Evaluation of Marker-Assisted Introgression of Yield QTL Alleles into Adapted Soybean.” Crop Sci. 41:1317-1321
Ribaut J-M, Bertran J. 1999. “Single large-scale marker-assisted selection (SLS-MAS).” Mol. Breeding 5:531-541
Ribaut J-M, Jiang C, Hoisington D. 2002. “Simulation Experiments on efficiencies of Gene Introgression by Backcrossing.” Crop Sci. 42:557-565
Rouppe van der Voort J N A, Zandvoort van P, Eck van H J, Folkertsma R T, Hutten R C B, Draaistra J, Gommers F J, Jacobsen E, Helder J, Bakker J. 1997. “Use of allele specificity of comigrating AFLP markers to align genetic maps from different potato genotypes.” Mol. Gen. Gen. 255:438-447.
Rungis D, Hamberger B, Berube Y, Wilkin J, Bohlmann J, Ritland K. 2005. “Efficient genetic mapping of single nucleotide polymorphisms based upon DNA mismatch digestion.” Mol. Breeding 16:261-270
Saha M C, Mian R, Zwonitzer J C, Chekhovskiy K, Hopkins A A. 2005. “An SSR- and AFLP-based genetic linkage map of tall fescue (Festuca arudinacea Schreb.).” Theor. Appl. Genet. 110:323-336
Saliba-Colombani V, Causse M, Langlois D, Philouze J, Buret M. 2001. “Genetic analysis of fruit quality in fresh market tomato. 1. Mapping QTLs for physical and chemical traits.” Theor. Appl. Genet. 102:259-272
Salmaso M, Faes G, Segala C, Stefanini M, Salakhutdinov I, Zyprian E, Toepfer R, Grando M S, Velasco R. 2004. “Genome diversity and gene haplotypes in the grapevine (Vitis vinifera L.) as revealed by single nucleotide polymorphisms.” Mol. Breeding 14:385-395
Sandal N, Krusell L, Radutoiu S, Olbryt M, Pedrosa A, Sracke S, Sato S, Kato T, Tabata S, Parniske M, Bachmair A, Ketelsen T, Stougaard J. 2002. “A genetic linkage map of the model legume Lotus japonicus and strategies for fast mapping of new loci.” Genetics 161:1673-1683
Santos C A F, Simon P W. 2004. “Merging Carrot Linkage Groups based on Conserved Dominant AFLP Markers in F2 Populations.” J. Am. Soc. Hort. Sci. 129:211-217
Sasaki T, Matsumoto T, Antonio B A, Nagamura Y. 2005. “From mapping to sequencing, post-sequencing and beyond.” Plant Cell Physiol. 46:3-13
Scalfi M, Troggio M, Piovani P, Leonardi S, magnaschi G, Vendramin G G, Menozzi P. 2004. “A RAPD, AFLP and SSR linkage map, and QTL analysis in European beech (Fagus sylvatica L.).” Theor. Appl. Genet. 108:433-441
Schneider K, Weisshaar B, Borchardt D, Salamini F. 2001. “SNP frequency and allelic haplotype structure of Beta vulgaris expressed genes.” Mol. Breeding 8:63-74
Sebastian, R L, Howell E C, king G J, Marshall D F, Kearsey M J. 2000. “An integrated AFLP and RFLP Brassica oleraceae linkage map from two morphologically distinct doubled-haploid mapping populations.” Theor. Appl. Genet. 100:75-81
Shi C, Thummler F, Melchinger A E, Wenzel G, Lubberstedt T. 2005. “Association between SCMV resistance and microarray-based expression patterns in maize inbreds.” Mol. Breeding 16:173-184
Shirasawa K, Kishitani S, Nishio T. 2004. “Conversion of AFLP markers to sequence-specific markers for closely related lines in rice by use of the rice genome sequence.” Mol. Breeding 14:283-292
Silberstein L, Kovalski I, Brotman Y, Perin C, Dogimont C, Pitrat M, Klingler J, Thompson G, Portnoy V, Katzir N, Perl-Treves R. 2003. “Linkage map of Cucumis melo including phenotypic traits and sequence-characterized genes.” Genome 46:761-773
Song O J, Marek L F, Shoemaker R C, Lark K G, Concibido V C, Delanney. X, Specht J E, Cregan P B. 2004. “A new integrated genetic linkage map of the soybean.” Theor. Appl. Genet. 109:122-128
Sorrels M E, Wilson W A. 1997. “Direct classification and selection of superior alleles for crop improvement.” Crop Sci. 37:691-697
Stam P. 1993. “Construction of integrated linkage maps by means of a new computer package: JoinMap. Plant J. 3:739-744
Strommer J, Peters J, Zethof J, De Keukeleire P, Gerats T. 2002. “AFLP maps of petunia hybrida: building maps when markers cluster.” Theor. Appl. Genet. 105:1000-1009
Stuber C W, Polacco M, Senior M L. 1999. “Synergy of empirical Breeding, Marker-Assisted Selection, and Genomics to Increase Crop Yield Potential.” Crop Sci. 39:1571-1583
Sullivan J G, Bliss F A. 1983. “Genetic Control of Quantitative Variation in Phaseolin Seed Protein of Common Bean.” J. Amer. Soc. Hort. Sci. 108:782-787
Sun G L. William M, Liu J, Kasha K J, Pauls K P. 2001. “Microsatellite and RAPD polymorphisms in Ontarion corn hybrids are related to the commercial sources and maturity ratings.” Mol. Breeding 7:13-24
Syed N H, Sorensen A P, Antonise R, van de Viel C, van der Linden C G, van't Westende W, Hooftman Da, den Nijs H C, Flavell A J. 2006 “A detailed linkage map of lettuce based on SSAP, AFLP, and NBS markers.” Theor. Appl. Genet. 112:517-527
Tang S, Leon A, Brodges W C, Knapp S J. 2006. “Quantitative Trait Loci for Geneticaly Correlated Seed Traits are Tightly Linked to Branching and Pericarp Pigment Loci in Sunflower.” Crop Sci. 46:721-734
Tanksley S D, Ganal M W, Prince J P, de Vicente M, Bonierbale M W, Broun P, Fulton T M, Giovannoni J J, Grandillo S, Martin G B, Messegure R, Miller J C, Miller, L, Paterson A H, Pineda O, Roder M S, Wing R A, Wu W, Young N D. 1992. “High Density Molecular Linkage Map of the Tomato and Potato Genomes.” Genetics 132:1141-1160.
Tanksley S D, Nelson J C. 1996. “Advanced backcross QTL analysis: a method for the simultaneous discovery and transfer of valuable QTLs from unadapted germplasm into elite breeding lines.” Theor. Appl. Genet. 92:191-203
Tanksley S D, McCouch S R. 1997. “Seed banks and Molecular Maps: Unlocking Genetic Potential from the Wild.” Science 277:1063-1066
Tekeoglu M, Rajesh N, Muehlbauer J. 2002. “Integration of sequence tagged microsatelite sites to the chickpea genetic map.” Theor. Appl. Genet. 105:847-854
Thomson M J, Tai T H, McClung A M, Lai X H, Hinga M E, Lobos K B, Xu Y, Martinez C P, McCouch S R. 2003. “Mapping quantitative trait loci for yield, yield components and morphological traits in an advanced backcross population between Oryza rufipogon and the Oryza sativa cultivar Jefferson.” Theor. Appl. Genet. 107:479-493
Tian F, Li D J, Fu Q, Zhu Z F, Fu Y C, Wang X K, Sun C Q. 2006. “Construction of introgression lines carrying wild rice (Oryza rufipogon Griff.) segments in cultivated rice (Oryza sativa L.) background and characterization of introgressed segments associated with yield-related traits.” Theor. Appl. Genet. 112:570-580
Torada A, Koike M, Mochida K, Ogihara Y. 2006. “SSR-based linkage map with new markers using an intraspecific population of common wheat.” Theor. Appl. Genet. 112:1-10
Tsaurouhas V, Gullberg U, Lagerkrantz U. 2002. “An AFLP and RFLP linkage map and quantitative trait locus (QTL) analysis of growth traits in Salix.” Theor. Appl. Genet. 105:277-288
Ulloa M, Meredith W R Jr., Shappley Z W, Kahler A L. 2002. “RFLP genetic linkage maps from four F(2.3) populations and joinmap of Gossypium hirsutum L.” Theor. Appl. Genet. 104:200-208
Van der Hoeven R, Ronning C, Giovannoni J, Martin G, Tanksley S. 2002. “Deductions about the Number, Organization, and Evolution of Genes in the Tomato Genome Based on Ananlysis of a Large Expressed Sequence Tag Collection and Selective Genomic Sequencing.” The Plant Cell 14:1441-1456
Varshney R K, Graner A, Sorrells M E. 2005. “Genomics-assisted breeding for crop improvement.” Trends Plant Sci. 10:621-630
Vilanova S, Romero C, Abbott A G, Llacer G, Badenes M L. 2003. “An apricot (Prunus armeniaca L.) progeny linkage map based on SSR and AFLP markers, mapping plum pox virus resistance and self-incompatibility traits.” Theor. Appl. Genet. 107:239-247
Wang D, Graef G L, Prokopiuk A M, Diers B W. 2004. “Identification of putative QTL that underlie yield in interspecific soybean backcross populations.” Theor. Appl. Genet. 108:458-467
Wehrhahn C, Allard R W. 1965. “The detection and measurement of the effects of individual genes involved in the inheritance of a quantitative character in wheat.” Genetics 51:109-119
Xiao J, Li J, Grandillo S, Ahn S N, Yuan L, Tanksley S D, McCouch S R. 1998. “Identification of Trait-Improving Quantitative Trait Loci Alleles From a Wild Rice Relative, Oryza rufipogon. Genetics 150:899-909
Xu J L, Lafitte H R, Gao Y M, Fu B Y. 2005. “QTLs for drought escape and tolerance identified in a set of random introgression lines of rice.” Theor. Appl. Genet. 111: 1642-1650
Yan Z, Donnenboom C, Hattendorf A, Dolstra O, Debener T, Stam P, Visser P B. 2005. “Construction of an integrated map of rose with AFLP, SSR, PK, RGA, RFLP, SCAR and morphological markers. Theor. Appl. Genet. 110:766-777
Yin T, Zhang X, Huang M, Wang m, Zhuge O, Tu S, Zhu L H, Wu R. 2002. “Molecular linkage maps of the Populus genome.” Genome 45:541-555
Yonezawa K, Ishii T. 2005. “Marker-based Selection as a Tool for Enhancing the Efficiency of Two Conventional Breeding Methods of Self-fertilizing Crop Plants, the Generation-accelerated Bulk Breeding and Doubled Haploid Breeding.” Breeding Sci. 55:397-407
Yu K, Park S J, Gepts P. 2000 “Integration of simple sequence repeat (SSR) markers into molecular linkage map of common bean (Phaseolus vulgaris L.)” J. Hered. 9:429-434
Zhang R, Xu Y, Yi K, Zhang H, Gong G, Levi A. 2004. “A genetic Linkage Map for Watermelon Derived from Recombinant Inbred Lines.” J. Am. Soc. Hort. Sci. 129:##
Zhang W K, Wang Y J, Luo G Z, Zhang J S, He C Y, Wu X L, Gai J Y, Chen S Y. 2004. “QTL mapping of ten agronomic traits on the soybean (Glycine max L. Merr.) genetic map and their association with EST markers. Theor. Appl. Genet. 108:1131-1139
Zhu S, Kaeppler H F. 2003. “A genetic linkage map for hekaploid, cultivated oat (Avena sativa L.) based on an intraspecific cross “Ogle/MAMI17-5”.” Theor. Appl. Genet. 107:26-35

3. Description of Related Art

During the past several years genetic maps have been developed for all major agronomic crops such as maize (Davis et al. 1999, Falque et al. 2005), rice (Chen et al. 2002), sorghum (Menz et al. 2002, Bowers et al. 2003, Feltus et al. 2006), wheat (Paillard et al. 2003, Quarrie et al. 2005, Liu et al. 2005, Torada et al. 2006)), oats (Kremer et al. 2001, Zhu and Kaeppler 2003, De Koeyer et al. 2004), barley (Ramsay et al. 2000, Hori et al. 2003), rye (Bednarek et al. 2003), potato (Rouppe van der Voort et al. 1997, Brugmans et al. 2006), cotton (Ulloa et al. 2002), sunflower (Gedil et al. 2001), rape seed (Piquemal J. et al. 2005), soybean (Cregan et al. 1999, Song et al. 2004), sugar cane (Garcia et al. 2006), coffee (Pearl et al. 2004), tea (Hackett et al. 2000) and cacao (Pugh et al. 2004); forage crops such as alfalfa ((Julier et al. 2003), red clover ((Isobe et al. 2003), and various grasses (Alm et al. 2003, Faville et al. 2004, Saha et al. 2005); vegetable crops such as lettuce (Kesseli et al. 1994, Syed et al. 2006), bean (Yu et al. 2000, Blair et al. 2003), pea (Aubert et al. 2006), mungbean (Humphry et al. 2002), chickpea (Tekeoglu et al. 2002), cowpea (Ouedraogo et al. 2002), lentil (Hamwieh et al. 2005); tomato (Tanksley et al. 1992), pepper (Livingstone et al. 1999, Paran et al. 2004, Ogundiwin et al. 2005), eggplant (Doganlar et al. 2002)), many species in the Brassicaceae family (Sebastian et al. 2000; Pradhan et al. 2003), muskmelon (Oliver et al. 2001, Perin et al. 2002, Silberstein et al. 2003), cucumber (Park et al. 2000, Bradeen et al. 2001), watermelon (Levi et al. 2002, Hashizume et al. 2003, Zhang et al. 2004), and carrots (Santos and Simon 2004); grapes (Lodhi et al. 1995, Doligez et al. 2002, Adam-Blondon et al. 2004); fruit trees such as peach (Dettori et al. 2001, Bliss et al. 2002), apricot (Vilanova et al. 2003), apple (Liebhard et al. 2003), almond (Joobeur et al. 2000), pecan (Beedanagari et al. 2005)), hazelnut (Mehlenbacher et al. 2006), and olive (la Rosa et al. 2003); forest trees such as willow (Hanley et al. 2002, Tsarouhas et al. 2002), white spruce (Gosselin et al. 2002), several species of poplar (Cervera et al. 2001, Yin et al. 2002), and beech (Scalfi et al. 2004); ornamentals such as roses, lily and petunia (Dugo et al. 2005, Yan et al. 2005, Abe et al. 2002, Strommer et al. 2002).

Most of the genetic maps were initially based on Restriction Fragment Length Polymorphism (RFLP) marker technology, which allows detection of gross differences, and thus is mostly applicable to comparisons between highly divergent genotypes such as between cultivated forms and their wild counterparts. Development of next generation markers, however, such as Amplified Fragment Length Polymorphism (AFLP), Randomly Amplified Polymorphic DNA (RAPD), Simple Sequence Repeats (SSR) and, ultimately, Single Nucleotide Polymorphism (SNP) markers, provided means for more detailed genetic comparisons. Subsequently, second generation genetic maps are being developed that incorporate AFLP, SSR and SNP markers in addition to the RFLP markers (Masi et al. 2003, Mohring et al. 2004, Salmaso et al. 2004, Shirasawa et al. 2004, Kenis and Keulemans 2005, Merdinoglu et al. 2005, Rungis et al. 2005). These markers provide a very high level of resolution of DNA polymorphism and can be used for detailed characterization of breeding germplasm (Schneider et al. 2001, Sun et al. 2001) and for elucidation of germplasm relatedness (Heckenberger et al. 2002, Heckenberger et al. 2003, Heckenberger et al. 2006).

The development of advanced genetic maps is being quickly followed by the development of physical maps through genome sequencing. In the United States the National Plant Genome Initiative (NPGI, www.ostp.gov/NSTC/html/npgi2003) was established in 1998 to coordinate efforts of Department of Agriculture (USDA), Department of Energy (DOE) National Institutes of health (NIH), National Science Foundation (NSF), Office of Science and Technology Policy (OSTP), and the Office of Management and Budget (OMB) in the area of genomic sequencing and genomic technology development.

The accomplishments of NPGI include the sequencing of the Arabidopsis genome, completion of a deep draft of rice genome, fundamental research discoveries, production of plant genome research resources, development of plant genome research tools, and establishment and participation in international collaborations: the Multinational Coordinated Arabidopsis thaliana Functional Genomics Project; the International Rice Genome Sequencing Project; the Cereal Genome Initiative; the International Genome Research Organization for Wheat; International Tomato Genome Sequencing Community; the Medicago truncatula Genome Group; the Poplar Functional Genomics Consortium; and the Global Musa Genomic Consortium. The development of plant research resources supported by NPGI includes the following: large collection of plant Expressed Sequence Tags (ESTs), Bacterial Artificial Chromosomes (BAC) libraries for over 72 plant species; a collection of transposon tagged lines; deep physical maps of maize, soybean, wheat and other plant species; and various public plant genomic databases. The development of plant genome research tools supported by NPGI provides key enabling technologies for genomic research. New research tools are being developed in the following areas: gene expression profiling tools including a whole-genome array for Arabidopsis; informatics tools to access, analyze, and synthesize all levels of plant genome data; and new optical mapping methods.

In addition to programs sponsored by NPGI initiatives are underway to sequence the genomes of other major crops, i.e. The Potato Sequencing Consortium, (http://potatogenome.net). Private organizations such as The Institute for Genomic Research (TIGR, www.tigr.org) provide additional resources. Public databases provide access to highly advanced maps and to a plethora of markers available for use (Maize Genomics Database www.maizegdb.org, Gramene—Cereal Comparative Mapping Database www.gramene.org, The Soybean Genome Database—www.soybeangenome.siu.edu, the SOL Genomics Network a comparative database for the Solanaceae family www.sgn.cornell.edu, a database for the Compositae species www.compositdb.ucdavis.edu).

The availability of physical sequence maps of selected species will in turn provide a basis for an in-depth understanding of genomic organization and development of highly refined maps of all economically important species through the identification of conserved ortholog sequences (COS) and inferences drawn from genomic synteny (Grant et al. 2000, Ku et al. 2000, Pan et al. 2000, Drave et al. 2001, Ku et al. 2001, Coe et al. 2002, Doganlar et al. 2002, Fourmann et al. 2002, Fulton et al. 2002, Sandal et al. 2002, Van der Hoeven et al. 2002, Babula et al. 2003, Frary et al. 2003, Ilic et al. 2003, Fridman and Zamir 2003, Zhang et al: 2004, Bauer et al. 2004, Rensink et al. 2005, Lin et al. 2005, Sasaki et al. 2005)

Furthermore, in addition to the rapidly developing marker and sequence information technology, very rapid progress is being made in developing tools for analysis of the whole genome. Recent development of microarrays, or “genetic chips”, provides an unprecedented capacity for large scale genomic comparisons (Alba et al. 2004, Asamizu et al. 2004, Shi et al. 2005, Moran et al. 2006, Meaburn et al. 2006).

Technological and computational platforms are being developed for large scale genetic analysis and mapping (Stam 1993, Nelson 1997, Manly et al. 2001, Cone et al. 2002, Fang et al. 2003 (a), Fang et all 2003(b)). Moreover, the National Human Genome Research Institute is sponsoring development of sequencing technology that will produce complete genome sequences at the cost of $1,000 each, thus enabling the whole genome analysis on routine bases (www.genome.gov). It is therefore clear that biological sciences and plant and animal breeding in particular will have a plethora of new tools available to them in the near future.

Development of genetic maps made possible formulation of methodologies for identification of quantitative trait loci (QTL) of economic importance. Several schemes have been proposed for QTL identification and mapping (Edwards et al. 1987, Paterson et al. 1988, Lander and Botstein 1989, Tanksley and Nelson 1996). In principle, the QTL methods can be divided into two groups, one based on F2 populations and/or utilizing the so-called pure F2 and F3 lines, and the other relying on a backcross scheme. The F2-line model is considered to have more power for QTL detection, however lines created in this model are a mix of both parents, and thus need to be evaluated de novo for utility in breeding. The backcross-based method has the practical advantage of retaining most of the breeding advantages of the recurrent parent.

Numerous QTL-identification programs have been executed, primarily by university researchers, in order to identify useful genetic variation in agronomically unadapted species. An example of this type of research is the work done in tomato and rice (Paterson et al. 1990, Paterson et al. 1991, deVicente and Tanksley 1993, Eshed and Zamir 1995, Bemacchi and Tanksley 1997, Tanksley and McCouch 1997, Xiao et al. 1998, Doganlar et al. 2002, Thomson et al. 2003, Frary et al. 2004, Tian et al. 2006). The same concept of breeding germplasm enrichment via QTL identification in wild or unadapted relatives has been applied to virtually all economically important crops such as soybeans, cotton, barley, and many others (Keim et al. 1990, Wang et al. 2004, Chee et al. 2005, Hori et al. 2005, Korff et al. 2005). To a lesser degree QTL mapping was performed in populations derived from crosses between cultivated forms (Causse et al. 2001, Reyna and Sneller 2001, Saliba-Colombani et al. 2001, Causse et al. 2002, Ho et al. 2002, Fischer et al. 2004, Huang et al. 2004, Xu et al. 2005, Mei et al. 2006, Tang et al. 2006)

The development and testing of a QTL-mapping population and the development of near-isogenic-lines (NIL) can take several years, before the results can be utilized by a collaborating commercial breeder (Monforte and Tanksley 2000, Monforte et al. 2001. Bouchez et al. 2002, Chaib et al. 2006). By this time the recurrent parent used in the population development is obsolete commercially, and the identified and purified QTLs need to be reintroduced into a competitive commercially germplasm via a time-consuming backcrossing scheme.

Marker application in plant breeding is limited by the available portfolio of markers. Development of marker-assisted selection (MAS) and the application of marker-assisted breeding (MAB) are very expensive, as they require costly laboratory equipment and supplies, and highly paid staff. Often, a business decision has to be made as to which genetic characteristics are of sufficient economic value to warrant the expense (Dreher et al. 2003, Morris et al. 2003, Kuchel et al. 2005).

Consequently, only large seed companies specializing in high cash-value crops such as corn or soybean can contemplate extensive application of MAS, MAB and QTL identification in product development. The methodologies used by these companies are often patented (U.S. Pat. No. 6,399,855).

The most frequently used mode of MAS application in smaller programs is a step-wise pyramiding of target characteristics. The most readily available molecular markers are the ones associated with characteristics that have clearly identifiable phenotypic expression. These characteristics used to be identified using functional assays, such as screening for disease resistance, for example. Thus, in the current scenario, the marker technology used by smaller companies is primarily a replacement technology.

The very rapid progress in the area of genome analysis has led to the recognition of the importance of integration of genomic technology into the activities of commercial breeders. The need to develop a synergistic relationship between breeding and genomic activities has been addressed in several publications (Lee 1998, Namkoong et al. 2004, Bonnett et al. 2005). Breeding schemes amenable to the use of marker technology have been reviewed (Sorrels and Wilson 1997, Stuber et al. 1999, Ribaut and Betran 1999, Ribaut et al. 2002, Charcosset and Moreau 2004, Collard et al. 2005, Francia et al. 2005, Varshney et al. 2005, Yonezawa and Ishii 2005), but the authors fall short of providing an integrated and clear blueprint that can be understood and implemented not only by a breeder but also by a business manager. And yet, the tools developed through the genomic initiatives need to be applied, verified, and integrated into commercial breeding on a large scale in order to take a full economic advantage of the massive expenditures associated with the development of genomic technology.

### SUMMARY OF THE INVENTION

A system and method for integration of commercial plant breeding and genomic technologies where the two platforms are combined and applied repeatedly to achieve complete integration.

More particularly, the method provides for simultanesous development of a breeding population with molecular marker development and gene mapping, and integration of the molecular marker platform with the breeding platform. The breeding population is developed through performing an initial cross, followed by two back-crosses and self-pollination of BC2F1 plants. Molecular marker development consists of QTL identification using BC2F2 family means, gene fine mapping and new marker development using bulked-segregant analysis.

The steps include: a) developing a plant population by crossing a Parent 1 and a Parent 2 to generate a Population I; b) crossing Parent 1 with individuals from Population I to generated a Population II; c) crossing Parent 1 with individuals from population II to generate a Population III; d) randomly selecting at least one plant per each line in Population III and collecting genetic material from the random plant; e) self pollinating selected plants from population III to generate a Population IV; f) evaluating and selecting plants of Population IV; and g) using selecting progeny plants of Population IV in test crosses for evaluating the potential to develop new commercial cultivars; where the genetic material in step d) is used to develop marker profiles of each plant to map QTL and major gene loci as part of the evaluation of plants in step f).

In one alternative embodiment, the marker is linked to the trait of interest through development of the marker profiles in step f).

In one preferred embodiment, in step d) the genetic material is plant tissue preserved from the at least one random plant, while in a different embodiment, in step d) the genetic material is purified DNA prepared from the at least one random plant.

In further preferred embodiment, in step f) BC2F2 family means are used to evaluated the plants in step f).

Preferably, in step g) genomic inferences about combining ability are made to develop an integrated genomic breeding platform using marker profiles. In one alternative embodiment, the marker profiles are used in further development of new commercial cultivars.

In one preferred aspect of the invention, Parent 2 is a plant line commonly used in breeding, which can be an inbred plant line, a commercial hybrid, a breeding line, a landrace, a heirloom variety and a non-cultivated relative of Parent 1. In a further preferred aspect, Parent 1 and or Parent 2 can be a genetically engineered plant.

In one alternative embodiment, Parent 1 and Parent 2 are plants used in commercial cultivation. Common commercial cultivation crops include crops that are grown for agronomic, forage, pasture, turf, orchard, forestry, vegetable, ornamental or medicinal purposes, or crops that are used in environmental remediation. Also contemplated by the invention is the breeding of industrial crops.

In one preferred embodiment, Parent 1 and Parent 2 are dicot plants. Dicot plants for use in the invention include, but are in no ways limited to, plants of the plant orders Apiales, Asterales, Austrobaileyales, Brassicales, Cariophyllales, Cucurbitales, Ericales, Fabales, Fagales, Gentianales, Geraniales, Lamiales, Laurales, Malpighiales, Malvales, Myrtales, Pinales, Ranunculales, Rosales, Sapindales, Saxifragales, Solanales and Vitales.

In another preferred embodiment, Parent 1 and Parent 2 are monocot plants. Monocot plants for use in the invention include, but are in no ways limited to, plants of the plant orders Arecales, Asparagales, Liliales, Poales and Zingiberales.

In step f) plants are preferably evaluated by identification of a plant phenotype, for instance, the phenotype of resistance to a plant pathogen. Plant pathogens may include viral diseases, bacterial diseases, fungal diseases, nematode diseases, insect pests, and combinations thereof.

In a different alternative embodiment, the phenotype consists of a physiological characteristic, such as salt tolerance, drought tolerance, cold tolerance, heat tolerance, rate of growth, rate of methabolite accumulation, turgidity, ripening characteristics, rate of photosynthesis, respiration, reproductive biology, seed viability, seed dormancy, germination dynamics, vernalization, bolting, levels and timing of gene expression, and other physiological processes.

In another aspect, the phenotype is a morphological characteristic. Morphological characteristic that can be used for identifying and evaluating plants include such characteristics as plant size, organ size, shape, branching, root structure, color, surface characteristics, texture, and plant architecture, though other characteristics may also be used.

In a different aspect, of the invention, the phenotype is a biochemical characteristic. Preferred biochemical characteristic that can be used in the phenotypic evaluation include the accumulation of a secondary metabolite, plant nutritional value, vitamin composition and content, carbohydrate composition and content, acid composition and content, fiber composition and content, cellulose composition and content, fat composition and content, wax composition and content, and protein composition and content.

In an alternative embodiment, the phenotype is an agronomic characteristic such as yield, field holding, lagging resistance, seed set, long shelf life, and storability.

Another preferred embodiment for phenotype evaluation includes characteristics relating to industrial processing. Industrial processing characteristics of various crops include juice and serum viscosity, peelability, fiber length, fiber strength, fiber structure, ethanol production capacity, digestibility, fermentability.

The above method links an uninterrupted flow of commercial product development with either a concurrent or deferred application of genomic methodology, enabling a flexible and economically sound integration.

### BRIEF DESCRIPTION OF THE DRAWINGS

Various exemplary embodiments of this invention will be described in detail, with reference to the following figures, wherein:

FIG. 1 provides a diagram for the model for integration of commercial breeding and genomic technology.

FIG. 2 provides the schematic illustration of fine mapping of the QTL conferring the T1_{A} characteristic derived from the recurrent parent.

FIG. 3 shows a schematic illustration of fine mapping of the QTL conferring the T5_{B} characteristic derived from the donor parent.

### DETAILED DESCRIPTION OF THE PREFERRED EMBODIMENT

The present invention provides a management platform that combines a highly effective method of plant breeding with simultaneous discovery of economically important genes and application of genomic tools. It allows rapid delivery of commercially competitive product (plant inbreds, hybrids, and open-pollinated varieties) and, at the same time, creates a bridge between conventional breeding methodology and genome-based breeding, thus allowing the user to transition smoothly into the new technological platform of genomics. Application of this management platform will ultimately lead to conversion from conventional breeding methodology into DNA-sequence-based breeding.

The management model can be used as a blueprint by commercial seed companies of any size. This model specifies methodology for building a common genetic platform for uninterrupted delivery of commercial product and development of understanding and application of genomic technology for gene discovery and manipulation. Preferably, in step g) genomic inferences about combining ability are made to develop an integrated genomic breeding platform using marker profiles. In one alternative embodiment, the marker profiles are used in further development of new commercial cultivars.

An effective management tool should be based on sound and verified research principles, comprehensive, widely applicable, flexible with regard to timing and the extent to which it can be applied and creates synergies.

The model presented here meets all of these criteria. It combines a highly effective and proven breeding methodology with marker development and application, gene discovery, and mapping. It allows the practitioner to proceed with commercial product development independently of the gene discovery phase without losing the opportunity to apply the gene discovery methodology at a later time. It also allows the practitioner to perform molecular marker analysis at any time during this process, giving both financial and strategic flexibility. Furthermore, the genomic services can be either performed in-house or outsourced to a service provider without loosing proprietary information, thus enabling the practitioner to select the most appropriate and cost-effective solution. This business model gives a chance of survival to smaller companies as it enables them to develop and use molecular markers inexpensively and to develop proprietary genomic knowledge that has a value in cross-licensing of enabling technology, thus guaranteeing their long-term survival.

This methodology is particularly applicable to breeding products where external appearance needs to conform to pre-established consumer preferences and has to be preserved during the breeding process. Therefore it is particularly relevant to breeding vegetable and ornamental species where either visual (such as color or shape) or sensory (such as flavor or texture) characteristics reflect customer preferences and need to be maintained. Rapid identification of genes influencing these characteristics can speed-up the breeding process. The use of markers will help to maintain these characteristics in the breeding germplasm pool without costly biochemical and sensory analysis. This methodology also allows rapid combination of key characteristics with other characteristics that appeal to the customer, thus providing high return on the investment.

Parent lines for use as starting material includes inbred plant lines, commercial hybrids, various established breeding lines, landraces, heirloom varieties and various non-cultivated relatives of the parents, including wild or natural types.

In some cases, one or more of the parents may be genetically engineered plants.

The methods can be applied to any of various well know agronomic crops, but also to forage, pasture, turf, orchard, forestry, vegetable, ornamental or industrial crops.

Other crops where the system and method may find particular use is in the breeding of crops grown for medicinal purposes and crops that are used in environmental rem ediation, for the rapid identification of markers linked to the important phenotypic characteristics providing the key genetics responsible for their valued traits.

The breeding and genomics integration platform is based on a combination of four concepts verified in practice: a method for detection and measurement of the effects of individual genes involved in quantitative inheritance proposed by Wehrhahn and Allard in 1964, an Advanced-Backcross (AB) method of QTL mapping published by Tanksley and Nelson in 1996, QTL identification using BC2F2 families (Moncada et al. 2001), and QTL analysis in advanced breeding materials (Causse et al. 2001, Reyna and Sneller 2001, Saliba-Colombani et al. 2001, Causse et al. 2002, Ho et al. 2002, Fischer et al. 2004, Huang et al. 2004, Xu et al. 2005, Mei et al. 2006, Tang et al. 2006). These concepts are modified, expanded and amended, and assembled into a unique and cohesive magement model.

The method for detection and measurement of the effects of individual genes involved in the inheritance of a quantitative character was first proposed by Wehrhahn and Allard in 1964. It was successfully applied in the transfer of genes encoding phaseolin content in common bean (Sullivan and Bliss 1983). Sullivan and Bliss named the procedure the Inbred-Backcross (IBC) method.

The IBC method consists of crossing one parental line (donor parent) with another parental line (recurrent parent) to produce F1 progeny. The F1 progeny is then crossed again with the recurrent parent to produce a backcross progeny BC1). About 60 randomly selected BC1 lines are then crossed again with the recurrent parent resulting in 60 BC2 populations. One, randomly selected, plant from each of the 60 BC2 populations is then allowed to self pollinate for at least three generations via the method of single seed descent, followed by evaluation for presence of the characteristics transferred from the donor parent.

An assumption is made that single seed descent method will produce lines that are homozygotic for the characteristics introduced from the donor parent. Since the probability of obtaining a homozygote from self-pollinating a heterozygote is only 25% (50% of a progeny is heterozygotic and 25% does not inherit the donor allele), this procedure gives the highest chance of 50% to propagation of heterozygotes, and an even chance of 25% to either fixing or loosing the donor allele. This procedure is time consuming as it requires at least six generations for creation of the breeding population from which breeding lines will be extracted. Once a homozygotic line is established it is not known whether the characteristic of interest is dominant or recessive, until further testcrosses are performed. Thus there is no immediate insight into the segregation patterns of individual characteristics.

In the model presented here the donor alleles are brought to homozygosity by self-pollinating approximately 200 BC2 plants and evaluating their progenies. Evaluation of BC2F2 progenies is very important as it allows phenotypic detection of recessive alleles. The mode of inheritance (dominant vs. recessive) of the donor alleles can be immediately inferred from the segregation ratio observed among the 12-14 plants in each BC2F2 family. The BC2F2 families provide also an invaluable resource for marker development and precise QTL mapping.

The phenotype can be an easily identifiable morphological characteristic. Morphological characteristic that are commonly evaluated by breeders of commercial crops include plant size, organ size, shape, branching, root structure, color, surface characteristics, texture, and plant architecture.

Another phenotype commonly considered by breeders is resistance to a plant pathogen, such as a viral disease, bacterial disease, fungal disease, nematode disease, insect pest, or resistance to some combination of those pathogens.

Other phenotypes that could be considered are phenotypes relating to a physiological characteristic, such as salt tolerance, drought tolerance, cold tolerance, heat tolerance, rate of growth, rate of methabolite accumulation, turgidity, ripening characteristics, rate of photosynthesis, respiration, reproductive biology, seed viability, seed dormancy, germination dynamics, vernalization, bolting, levels and timing of gene expression, and other physiological processes.

Certain biochemical characteristics can also be evaluated. Biochemical characteristic frequently considered in breeding include the accumulation of a secondary metabolite, plant nutritional value, vitamin composition and content, carbohydrate composition and content, acid composition and content, fiber composition and content, cellulose composition and content, fat composition and content, wax composition and content, and protein composition and content.

Agronomic characteristic, such as yield, field holding, lagging resistance, seed set, long shelf life, and storability, are also commonly evaluated, and can be the basis of the phenotype evaluated by the disclosed method.

For certain other plants, phenotypic evaluation may relate more to characteristics relevant to industrial processing. Industrial processing characteristics of crops may include juice and serum viscosity, peelability, fiber length, fiber strength, fiber structure, ethanol production capacity, digestibility, fermentability.

The breeding integration method proposed here incorporates the QTL mapping protocol essentially as proposed by Tanksley and Nelson in 1996, however, no early selection is performed. This methodology, called Advanced-Backcross QTL mapping (AB QTL) was designed to facilitate identification and transfer of valuable QTLs from wild tomato species where an early selection against off-type plants was necessary as many of the BC1 and BC2 plants were either sterile or otherwise horticulturally unacceptable. The early selection creates gaps in the dispersal of the entire donor genome, thus limiting the number of inferences that can be made. In the Tanksley and Nelson model selected near-isogenic lines (NILs) carrying valuable QTLs are extracted and brought to homozygosity with the aid of molecular markers. The resulting fixed lines represent only a subset of the original population.

In the presented here model the QTL mapping is performed using BC2F2 family means. Similar approach to QTL identification was used successfully by Moncada et al. (2001) in rice. The use of BC2F2 family means allowed identification of novel QTLs in a cross between wild and cultivated forms of rice, however, their ultimate utility in rice breeding remains unknown as the plant population was not adapted agronomically.

The key feature of the model presented here is the simultaneous application of an effective breeding methodology, QTL identification methodology, marker development, and fine mapping of loci of interest. This methodology is comprehensive, economical, and provides key strategic advantages.

As mentioned above, this methodology allows detection of recessive characteristics that were transferred from the donor parent. The knowledge of the mode of inheritance of a given characteristic is very important in selecting parental lines for making a hybrid as a desirable recessively inherited characteristic will need to be present in both parental inbreds in order for a hybrid to express this characteristic as well. On the other hand, if deleterious characteristics are transferred from the donor parent through genetic linkage to a beneficial QTL, but they are inherited as recessive genes, they will not affect the performance of the hybrid product.

Another key advantage is the high degree of homozygosity of the tested material. Generation of a BC2 population reduces the average presence of the donor parent DNA to 12.5% of the entire genome. This means that on average only 12.5% of the genome is heterozygous as the 87.5% of the sequence that was inherited from the recurrent parent is homozygous.

Generation of BC2F2 families through self-pollination of BC2F1 plants further increases the homozygosity of the plants, thus selection among plants within the BC2F2 families will produce breeding lines that are highly homozygotic and can be used in preliminary test-crosses. One skilled in the art will also recognize that alternate approaches of can yield a similar genetic background. One such approach is a sib or sib-cross, referring to a cross of sibling plants.

The complete removal of all residual heterozygosity can be achieved by selection in one or two additional generations. The removal of the residual heterozygosity can be performed in parallel to the test-crosses allowing rapid discovery and delivery of commercial hybrid combinations.

Another key advantage of this model is the opportunity for a new marker development. Many breeding programs lack molecular markers that are linked to important commercially characteristics, thus enrichment of the molecular marker portfolio is always desirable. The BC2F2 lines represent an ideal material for identifying molecular markers associated with the characteristics that are derived from either one of the two parents using the method of bulked segregant analysis (Michelmore et al. 1991). The BC2F2 lines can be bulked into two groups, one containing lines with the characteristic of interest, the other containing lines without the characteristics. Sequence polymorphisms that are associated with the characteristic can be then identified by comparing polymorphic patterns of these two bulks. The identified polymorphic markers can be further re-screened using bulks of plants selected from within the BC2F2 families in which the characteristic segregated. Because different BC2F2 plants that inherited the characteristic of interest also inherited a different amount of linked donor DNA, the polymorphic marker that is identified in all BC2F2 plants expressing the characteristic should be closely linked to the targeted characteristic. This scenario is similar to the approach described for high resolution mapping of QTL (Peleman et al. 2005).

It is likely that the identified markers will be allele-specific and thus will provide a highly refined molecular tool for germplasm characterization. An additional advantage of the bulked segregant analysis is that it can be used to develop RAPD markers, which are inexpensive and the most straight-forward to use, thus they are ideal for a business with minimum experience and funding.

The BC2F2 population can be used for refining a QTL map, since additional molecular markers can be developed using a bulked-segregant method and then used in QTL analysis. The BC2F2-based marker development and QTL-mapping effort is especially powerful as it allows identification and mapping of recessive characteristics.

Further QTL mapping can be done using selected BC2F2 individulas. The various uses of backcross lines in QTL mapping have been extensively reviewed by others (Hill 1998, Hospital 2005). QTL identification can be a starting point for fine mapping, sequencing, and gene cloning. The ability to map valuable characteristics to specific chromosomal regions allows informatics-based predictions as to the types of genes governing these characteristics.

The ability to understand the mechanisms underlying hybrid vigor and to predict superior hybrid performance is of paramount importance to commercial breeders. The testcrosses between lines selected from the scheme presented here and various unrelated inbreds will provide very valuable information, as sets of introgression lines containing different genes combined in different genetic backgrounds can be used to study epistatic interactions (Hospital 2005). Furthermore selected lines can be used in a recurrent backcrossing scheme and further QTL identification (Hill 1998).

The practitioner of this invention will need to test a large number of different populations in order to create sufficiently large knowledge base to achieve full integration of commercial breeding and genomic technology. However, this model enables the practitioner to enter the realm of integration of breeding and genomics without loss of productivity. This model allows the practitioner to develop tools critical for gene discovery. These tools can be then applied to different breeding methodologies and to verification of new concepts.

A successful breeding platform model cannot overlook the effect that these procedures will have on the dynamics among the involved research staff. The benefits offered by this model to the breeders in the form of a commercial product and to the molecular biologists in the form of highly structured and well characterized populations create a tremendous level of synergy and mutual appreciation. The created plant populations offer an ideal platform for QTL and gene identification and for marker development, thus enabling a further deepening of molecular expertise. At the same time, maintaining focus on valuable commercially characteristics and a rapid delivery of commercial product stimulates collaborative will among the participants.

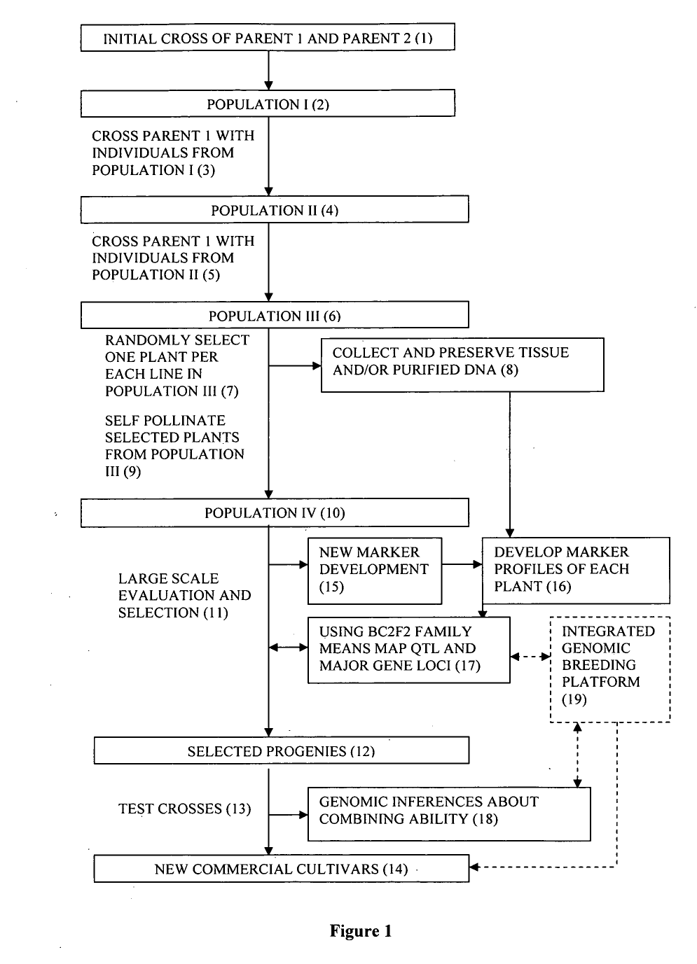

The breeding and genomics integration platform is represented schematically in FIG. 1. It consists of 19 steps, of which the first 14 steps are performed by the breeders with delivery of a commercial product at the end of the 14^{th} step. Marker development, QTL mapping and sophisticated genomic analysis are performed in steps 15-18, leading to integration of genomic research and future breeding efforts (Step 19). In order to arrive at Step 19 this model needs to be applied extensively to divergent genetic pools used in breeding of a given species in order to accumulate extensive genetic and genomic knowledge. Steps based on principles verified by research findings are drawn using solid lines. Since the final Step 19 is an inferred outcome it is drawn in a broken line.

A company that has no technological platform to perform molecular marker analysis but would like to be able to apply these methods in the future either in-house or by outsourcing needs only to perform the first 14 steps in order to maintain the strategic option of adding the remaining steps in the future. A company that is ready to apply marker analysis but does not have a sufficiently developed portfolio of molecular markers will need to complete steps 1 through 15 in order to acquire this strategic capacity. The completion of all steps gives the practitioner a full capacity of developing commercially competitive products with concomitant gene mapping and integration with genomic technologies.

The method can be applied with any of various types plants of interest, including both monocot and dicot crop varieties. For instance, the methods may be applied to plants of the order Apiales, consisting of, but not limited to, ginseng, carrot, and celery. Other dicot orders include Austrobaileyales, consisting of, but not limited to star anise, Brassicales, consisting of, but not limited to broccoli, cabbage, rapeseed and radish, Cariophyllales, consisting of, but not limited to beet, sugar beet, spinach and buckwheat, and Cucurbitales, consisting of, but not limited to plants such as cucumber, melon, waremelon, squash, pumpkin and begonia.

The method may also be applied to dicot orders such as Ericales, consisting of, but not limited to impatiens, primrose, tea, camellia, cranberry, blueberry and azalea, Geraniales, consisting of, but not limited to geranium, Gentianales, consisting of, but not limited to coffee, gardenia, periwinkle and oleander, and Fabales consisting of, but not limited to bean, clover, alfalfa and soybean.

The method may also be applied in the development of a breeding platform for plants of the dicot orders Asterales, consisting of, but not limited to sunflower, lettuce and artichoke, Malvales, consisting of, but not limited to cacao, cotton okra and mallow, and Ranunculales, consisting of, but not limited to anemone, delphinium and poppy.

The method may also be used in breeding plants of orders that include valuable tree crops, such as plants of the dicot order Fagales, which includes, but is not limited to, beech, walnut, pecan, birch and alder, the order Lamiales, consisting of, but not limited to olive, ash, basil, mint, oregano and foxglove or the order Laurales, consisting of, but not limited to avocado, cinnamon and laurel. Other orders include Rosales, consisting of, but not limited to almond, apple, apricot, peach, rose, raspberry, pear, plum, hemp, hops, fig and mulberry, Malpighiales, consisting of, but not limited to aspen, cottonwood, poplar, willow, violet, flax and cassaya, Myrtales, consisting of, but not limited to eucalyptus, myrtle and clove, Pinales, consisting of, but not limited to pine, spruce, cypress and yew, and Sapindales, consisting of, but not limited to lemon, orange, mango and maple.

Plants of other dicot crop orders may be used as Parent 1 and Parent 2, for instance, plants of the orders Saxifragales, consisting of, but not limited to black currant and goosbery, Solanales consisting of, but not limited to sweet potato, tomato, potato, pepper, eggplant, petunia and tobacco, and Vitales, consisting of, but not limited to grape.

The method of can also be applied to plants of a monocot crop, for instance plants belonging to the order Poales, consisting of, but not limited to bamboo, maize, rice, wheat, barley, oats and sugar cane. Other monocot plants that may be used with the method include plants of the orders Asparagales, consisting of, but not limited to orchid, leek, onion, and asparagus, Liliales, consisting of, but not limited to lily, tulip and crocus, Arecales, consisting of, but not limited to coconut and palm, and Zingiberales, consisting of, but not limited to banana and ginger.

### EXAMPLE 1

The initial cross can be made either between two inbreds or an inbred and a hybrid variety. It is important to select as Parent 1 an inbred of known good combining ability and commercial value that is actively being used in commercial product development. Parent 2 should be selected on the basis of highly desirable characteristics that are not observed in Parent 1. It is preferable that Parent 2 is derived from a distinctly different breeding pool.

A cross between two inbreds will produce genetically uniform (F1) Population I. In such case Population I can consist of as little as a single F1 plant. If Parent 2 is a hybrid variety then the resulting Population I will be genetically heterogeneous. In this case it is important that Population I consists of about 200 plants in order to ensure as complete as possible representation of Parent 2 alleles in the progeny.

Next step is a backcross step in which the F1 progeny (Population I) is crossed back to Parent 1 (the recurrent parent) to create Population II. If Population I consists of genetically uniform F1 plants only one F1 plant needs to be crossed with Parent 1. If Population I is heterogeneous, each of the ˜200 plants has to be crossed individually to Parent 1.

Population II is a BC1 population. If two inbreds were used in the initial cross it consists of 200 plants that are the progeny from a cross between one F1 plant and the recurrent parent. If an inbred and a hybrid variety were crossed initially, Population II consists of 200 plants, where each plant is derived from a different individual cross-pollination.

A second backcross to the recurrent parent is performed. Parent 1 is being crossed individually with each of the ˜200 plants in Population II to create Population III.

Population III is a BC2 population. It consists of 200 lines.

One plant is randomly selected per each BC2 line in Population III.

Tissue is collected from plants selected in Population III and preserved through freeze-drying or used for DNA extraction. The tissue and/or DNA is stored for use in molecular marker analysis.

Each selected in Population III BC2 μl plant is self-pollinated.

Population IV consists of 200 BC2F2 lines that resulted from self-pollinating BC2F1 plants in Population III.

Between 12 and 24 plants per each of the ˜200 BC2F2 lines are evaluated for the desired characteristics and selections are made.

Progenies of selected plants are grown out in replication and selected again for uniformity and stability of performance over different environments.

Test-crosses between selected lines and inbreds known for good combining ability with Parent 1 are made and evaluated in an appropriate environment.

New commercial cultivars are identified based on performance of the testcrosses.

New molecular markers are identified through bulked-segregant analysis.

DNA obtained from plants in Population III is analyzed for polymorphism.

Mean values of phenotypic characteristics in each BC2F2 family are correlated with the marker data using standard QTL-mapping procedures to identify valuable QTLs and major gene loci.

Test-cross information is used to draw inferences about genetic composition of the selected lines and their performance in hybrid combinations.

The long-term application of this model allows the rapid integration of marker, genomics and breeding technologies.

### EXAMPLE 2

A cross is made between a fresh market tomato inbred “A” and an open pollinated heirloom variety “B”. In addition to having an overall balanced and commercially acceptable phenotype, inbred A has three highly desirable traits (T1_{A}-T3_{A}) that need to be preserved in the process of breeding, and two highly undesirable characteristics (T4_{A} and T5_{A}) that need improvement. This inbred has a history of being used in commercial product development and is known to produce high yielding commercial hybrids with inbreds X, Y and Z. Parent B has no commercial value but is superior to Parent A in the two characteristics for which Parent A is deficient. The two valuable characteristics of parent B are designated as T4_{B} and T5_{B}.

The objective is to obtain inbred lines essentially of the type A Parent that will contain both desirable traits from Parent B. Another equally important objective is to develop molecular markers closely linked to all five characteristics for future use in marker assisted breeding, germplasm identification and protection, and whole genome scans using microarrays for identification of variation among alleles encoding these characteristics.

Due to the high cost of developing assays and identifying polymorphic markers only 90 molecular markers (M1-M90) that differentiate Parent A from Parent B are identified using publicly available genetic maps and marker sequences for tomato. It is not known whether the identified markers are linked to the characteristics of interest. The average distance between the developed markers is 15-20 cM, allowing inference about gross association of molecular markers and plant characteristics, but insufficiently dense for use in marker assisted breeding.

The characteristics T1-T5 can be evaluated either by performing measurements (traits that are amenable to metric evaluation) or by ranking plants using a scale developed by the breeder. Table 1 shows the average expression of targeted phenotypic characteristics in Parents A and B.

TABLE 1

| Characteristic | Parent A | Parent B |
|---|---|---|
| T1 | 4 | 2 |
| T2 | 10 | 5 |
| T3 | 120 | 52 |
| T4 | 2 | 5 |
| T5 | 4.8 | 6.4 |

Parent A is crossed with Parent B. Because both parents are homozygotic the F1 progeny is genetically uniform. Therefore only one F1 plant is crossed to Parent A to produce the first backcross (BC1). A random sample of 180 seeds is used to grow 180 BC1 plants, which are then crossed with Parent A to produce 180 BC2 lines. One seed per each BC2 line is randomly selected and grown into a plant that is self-pollinated. DNA is extracted from each plant, purified, and assayed for presence of introgressions from Parent B. The self-pollination of the 180 BC2 plants results in 180 BC2F2 families. Throughout the process seeds are selected randomly and there is no selection among the plants.

Twelve randomly selected seeds are sown per each BC2F2 family. Resulting plants are transplanted to the field and evaluated individually for the targeted characteristic. The average value for each characteristic is determined for each BC2F2 family.

QTL analysis is performed using BC2F2 family average values and the corresponding BC2F1 plant genotypes essentially as described in the literature (Tanksley and Nelson, 1996). Shown in Table 2 is an example of marker/phenotype associations typically found in a QTL analysis.

TABLE 2

| | | | | Inferred | |
|---|---|---|---|---|---|
| | | | | mode of | Superior |
| Characteristic | Marker | A/A^{a)} | A/B | inheritance | parent |
| T1 | M16–M17 | 4 | 4 | dominant | A |
| T2.1 | M75 | 8 | 6.5 | additive | A |
| T2.2 | M42–M44 | 6 | 5.5 | additive | A |
| T3.1 | M32–M33 | 84 | 84 | dominant | A |
| T3.2 | M20–M21 | 61 | 56 | additive | A |
| T4 | QTL not | — | — | — | B |
| | detected | | | | |
| T5 | M5–M6 | 4.8 | 5.6 | additive | B |

^{a)}Calculated average phenotypic value for a given genotype.

Trait-specific markers are developed either by fine-mapping of the identified QTLs, or by the method of bulked-segregant analysis.

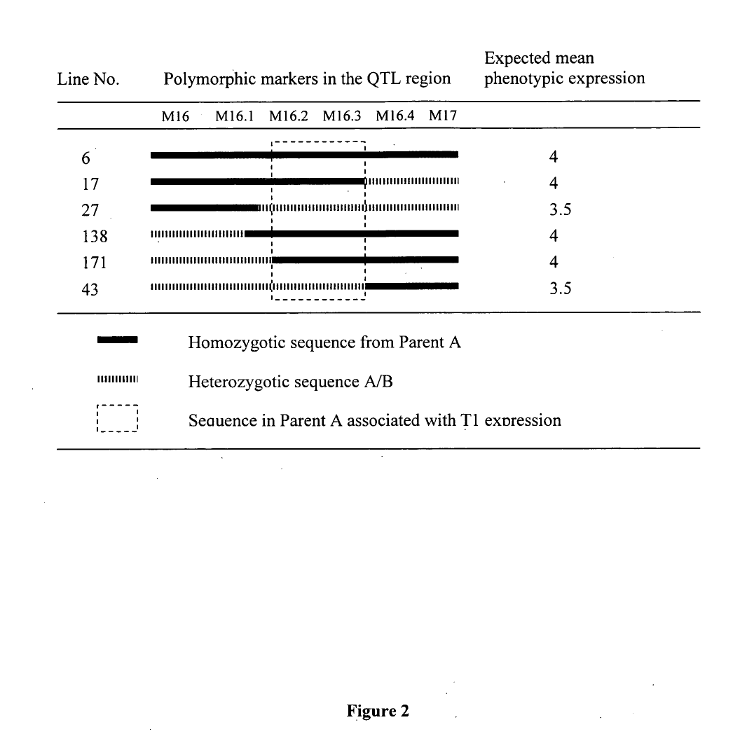

The procedure for fine mapping of the QTL T1 which is derived from parent A is shown in FIG. 2. In backcross populations plants containing the QTL T1^{A} either are homozygous (expressing the recurrent parent phenotype) or are heterozygous if an introgression from Parent B is present. Therefore two average BC2F2 family values for T1^{A} are expected—an average of 4 in families homozygotic for T1^{A}, and an average of 3.5 in families that were derived from BC2F1 plants that contain allele T1_{B}. The QTL mapping indicates that markers M16 and M17 delineate the region where the QTL is located. Additional markers saturating the M16-M17 region are identified using publicly available databases and the T1_{A} QTL is fine mapped using BC2F2 family means.

Two QTLs for T2_{A} are identified: a major QTL T2.1_{A} and a lesser QTL T2.1_{A}. Fine mapping of T2.1_{A} is not needed as a single marker M75 is associated with this QTL. T2.2_{A} maps to a large region and confers a lesser effect, therefore it is not being fine mapped.

Two QTLs are identified for the characteristic T3—a major dominant QTL T3.1_{A} and a lesser additive QTL T3.2_{A}. Near isogenic lines are extracted from BC2S2 families containing the T3.1_{A} and T3.2_{A} QTLs with the aid of the existing markers. Additional markers saturating the QTL regions are identified using publicly available maps. Each QTL is then fine-mapped using the procedure described by Monforte et al. (2001).

A marker/phenotype association is not found for the trait T4_{B}. The failure to detect significant association between T4_{B} and molecular markers is likely due to lack of sufficient marker density. It is observed, however, that five BC2F2 families express the T4 characteristic with an average value of 2.8 indicating a presence of a recessive gene derived from Parent B. The segregation pattern observed among the BC2F2 plants shows a ratio of 3:1 with a quarter of the plants expressing the T4 characteristic at the Parent B level, which confirms the presence of a single recessive gene. The above approach of increasing marker density for a more precise mapping of this QTL is impractical since these families contain a number of various introgressions. Therefore, the bulked-segregant analysis is used to develop a RAPD marker for the gene encoding T4_{B}.

Two DNA bulks, one extracted from plants expressing T4_{B} and the other one from plants not expressing T4_{B} are screened against a panel of 100 pairs of random primers. This results in identification of 3 primer pairs that give a polymorphic banding pattern. Next, DNA from individual plants expressing T4_{B} is assayed using the three polymorphic markers. This results in identification of one primer pair that gives polymorphic banding pattern in all plants expressing T4_{B}. This primer pair is used for the RAPD assay for T4_{B} detection.

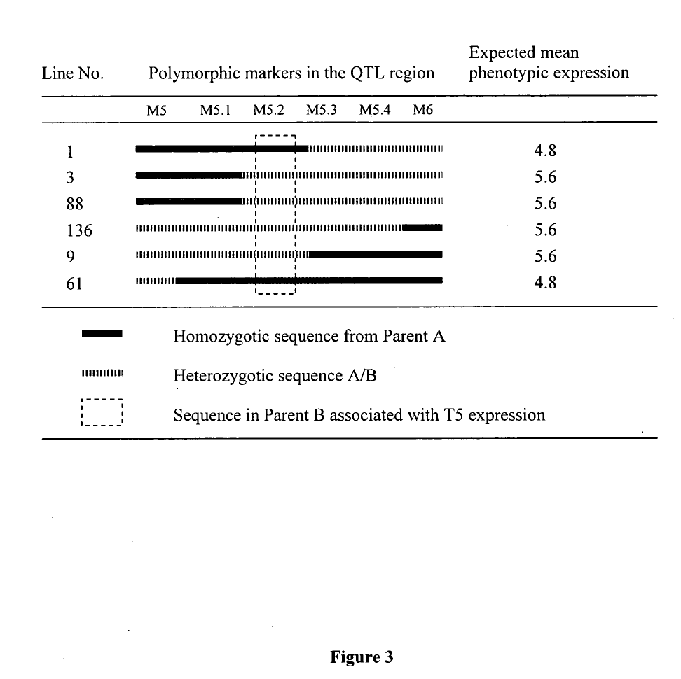

The QTL T5_{B} targeted for transfer from Parent B is fine-mapped by comparing levels of expression in all lines containing introgressions derived from Parent B in the M5-M6 region. The allele from Parent A is homozygous in all families except for families where T5_{B} was introduced through a crossover. Lines homozygotic for allele T5_{A} have an average expression of 4.8, whereas families derived from BC2F1 plants heterozygotic for T5_{B} have an average expression of 5.6. By identifying the smallest common region from Parent B the QTL T5 is located with high precision. Schematic representation of fine mapping of the T5_{B} QTL is shown in FIG. 3.

Detailed field observations of individual plants in the BC2F2 families allow selection of individual plants containing the desirable traits and having the best combination of all other characteristics. BC2F2 plants expressing Parent A level of T1, T2 and T3 are prioritized. This is possible because the backcross scheme results in high level of homozygosity of alleles derived from Parent A even though the QTL analysis shows that traits T1 and T2 are inherited as polygenic. Shown in Table 3 are the expected means of phenotypic descriptors of a BC2F2 family from which selections are made. Putative selections are shown in bold.

TABLE 3

| Plant No. | E(T1) | E(T2) | E(T3) | E(T4) | E(T5) |
|---|---|---|---|---|---|
| 1 | 4 | 10 | 120 | 5 | 5.6 |
| 2 | 4 | 10 | 120 | 2 | 5.6 |
| 3 | 4 | 10 | 120 | 5 | 4.8 |
| 4 | 4 | 10 | 120 | 2 | 6.4 |
| 5 | 4 | 10 | 120 | 5 | 4.8 |
| 6 | 4 | 10 | 120 | 2 | 6.4 |
| 7 | 4 | 10 | 120 | 2 | 5.6 |
| 8 | 4 | 10 | 120 | 2 | 6.4 |
| 9 | 4 | 10 | 120 | 2 | 4.8 |
| 10 | 4 | 10 | 120 | 2 | 5.6 |
| 11 | 4 | 10 | 120 | 2 | 5.6 |
| 12 | 4 | 10 | 120 | 2 | 5.6 |

Seeds from the selected plants are sown. Progenies need to be screened with markers to identify individuals homozygous for the targeted alleles. Progeny of plant 1 are screened with the M5.2 marker to identify plants homozygous for T5_{B}. Progenies of plants 4 and 8 are screened with the RAPD marker developed for detection of the gene conferring T4 in order to determine whether the genotypes of these plants are T4_{A}/T4_{A} or T4_{A}/T4_{B}. If the latter is true, plants homozygous for T4_{B} allele can be identified. BC2F3 plants homozygous for the targeted alleles are used for testcrosses with inbreds X, Y and Z. The best commercial hybrid combinations are then identified.

While this invention has been described in conjunction with the specific embodiments outlined above, it is evident that many alternatives, modifications and variations will be apparent to those skilled in the art. Accordingly, the preferred embodiments of the invention, as set forth above, are intended to be illustrative, not limiting. Various changes may be made without departing from the spirit and scope of this invention.

What is claimed is:

1. A method for the integration of commercial breeding and genomic technology to integrate a trait of interest from a donor parent, the method comprising the steps of:
a) making an initial cross of first Parent 1 and second Parent 2 to generate a Population I;
b) crossing Parent 1 with individuals from Population I to generated a Population II;
c) crossing Parent 1 with individuals from population II to generate a Population III;
d) randomly selecting at least one plant per each line in Population III and collecting genetic material from said random plant;
e) self pollinating selected plants from population III to generate a Population IV;
f) evaluating and selecting plants of Population IV; and
g) using selecting progeny plants of Population IV in test crosses for evaluating the potential to develop new commercial cultivars;
wherein said genetic material in step d) is used to develop marker profiles of each plant to map QTL and major gene loci as part of the evaluation of plants in step f).

2. The method of claim 1 wherein in step d) said genetic material is plant tissue preserved from said at least one random plant.

3. The method of claim 1 wherein in step d) said genetic material is purified DNA prepared from said at least one random plant.

4. The method of claim 1 wherein in step f) BC2F2 family means are used to evaluated plants in step f).

5. The method of claim 1 wherein step g) genomic inferences about combining ability are made to develop an integrated genomic breeding platform using marker profiles.

6. The method of claim 5 wherein said marker profiles are used in further development of new commercial cultivars.

7. The method of claim 1 wherein said marker is linked to said trait of interest through development of said marker profiles in step f).

8. The method of claim 1 wherein Parent 2 is a plant line commonly used in breeding.

9. The method of claim 8 where Parent 2 is a plant of a type selected from the group consisting of an inbred plant line, a commercial hybrid, a breeding line, a landrace, a heirloom variety and a non-cultivated relative of Parent 1.

10. The method of claim 1 wherein Parent 1 is a genetically engineered plant.

11. The method of claim 1 wherein Parent 2 is a genetically engineered plant.

12. The method of claim 1 wherein Parent 1 and Parent 2 are plants used in commercial cultivation.

13. The method of claim 12 wherein Parent 1 and Parent 2 are selected from the group of commercial cultivation crops consisting of agronomic, forage, pasture, turf, orchard, forestry, vegetable, ornamental, medicinal, environmental remediation or industrial crops.

14. The method of claim 1 wherein Parent 1 and Parent 2 are dicot plants.

15. The method of claim 14 wherein Parent 1 and Parent 2 are dicot plants of an order selected from the group of orders consisting of Apiales, Asterales, Austrobaileyales, Brassicales, Cariophyllales, Cucurbitales, Ericales, Fabales, Fagales, Gentianales, Geraniales, Lamiales, Laurales, Malpighiales, Malvales, Myrtales, Pinales, Ranunculales, Rosales, Sapindales, Saxifragales, Solanales and Vitales.

16. The method of claim 1 wherein Parent 1 and Parent 2 are monocot plants.

17. The method of claim 16 wherein Parent 1 and Parent 2 are monocot plants of an order selected from the group of orders consisting of Arecales, Asparagales, Liliales, Poales and Zingiberales.

18. The method of claim 1 wherein in step f) plants are evaluated by identification of a plant phenotype.

19. The method of claim 18 wherein said phenotype is resistance to a plant pathogen.

20. The method of claim 19 wherein said pathogen is selected from the group consisting of a viral disease, a bacterial disease, a fungal disease, a nematode disease an insect pest, and combinations thereof.

21. The method of claim 18 wherein said phenotype consists of a physiological characteristic.

22. The method of claim 21 wherein said physiological characteristic is selected from the group consisting of salt tolerance, drought tolerance, cold tolerance, heat tolerance, rate of growth, rate of methabolite accumulation, turgidity, ripening characteristics, rate of photosynthesis, respiration, reproductive biology, seed viability, seed dormancy, germination dynamics, vernalization, bolting, levels and timing of gene expression, and other physiological processes.

23. The method of claim 18 wherein said phenotype is a morphological characteristic.

24. The method of claim 23 wherein said morphological characteristic is selected from the group consisting of plant size, organ size, shape, branching, root structure, color, surface characteristics, texture, and plant architecture.

25. The method of claim 18 wherein said phenotype is a biochemical characteristic.

26. The method of claim 25 wherein said biochemical characteristic is selected from the group consisting of accumulation of a secondary metabolite, nutritional value, vitamin composition and content, carbohydrate composition and content, acid composition and content, fiber composition and content, cellulose composition and content, fat composition and content, wax composition and content, and protein composition and content.

27. The method of claim 18 wherein said phenotype is an agronomic characteristic.

28. The method of claim 27 wherein said agronomic characteristic is selected from the group consisting of yield, field holding, lagging resistance, seed set, long shelf life and storability.

29. The method of claim 18 wherein said phenotype is industrial processing characteristic.

30. The method of claim 29 wherein said industrial processing characteristic is selected from the group consisting of juice and serum viscosity, peelability, fiber length, fiber strength, fiber structure, ethanol production capacity, digestibility, fermentability.
